(19) United States
(12) Patent Application Publication
(10) Pub. No.: US 2007/0196490 A1
(43) Pub. Date: Aug. 23, 2007

(54) Method of enhancing needleless transdermal powered drug delivery

(75) Inventors: Sung-Yun Kwon, Fremont, CA (US); Terry L. Burkoth, Palo Alto, CA (US)

Correspondence Address: FOLEY AND LARDNER LLP;SUITE 500, 3000 K STREET NW, WASHINGTON, DC, 20007, US

(73) Assignee: POWDERJECT RESEARCH LIMITED

(21) Appl. No.: 11/488,192

(22) Filed: Jul. 18, 2006

Related U.S. Application Data
(63) Continuation of application No. 09/489,088, filed on Jan. 21, 2000, now abandoned.
(60) Provisional application No. 60/116,907, filed on Jan. 22, 1999.

(51) Int. Cl. A61K 9/38; A61K 9/14

(52) U.S. Cl. 424/477; 424/489

(57) ABSTRACT

A method is provided for enhancing the transdermal or intradermal delivery of a therapeutic agent using a needleless syringe drug delivery system. In one embodiment, the method entails first administering to a predetermined area of skin or mucosa particles comprising the therapeutic agent, a placebo and/or a permeation enhancing agent. A transdermal delivery device or occlusive dressing is then topically positioned over the area of skin or mucosa. In another embodiment, the method entails administering a formulation of particles wherein the formulation includes particles that contain a therapeutic agent and placebo particles.

### CROSS-REFERENCE TO RELATED APPLICATION

This application is related to provisional patent application serial no. 60/116,907, filed Jan. 22, 1999, from which priority is claimed under 35 USC §119(e)(1) and which is incorporated herein by reference in its entirety.

### TECHNICAL FIELD

The present invention relates generally to methods for administering a therapeutic agent to a subject. More particularly, the invention relates to methods for enhancing delivery of a therapeutic agent into vertebrate tissue using needleless powder injection techniques.

### BACKGROUND

The ability to deliver agents into and through skin surfaces (transdermal delivery) provides many advantages over other parenteral delivery techniques. In particular, transdermal delivery provides a safe, convenient and noninvasive alternative to traditional administration systems, conveniently avoiding the major problems associated with traditional needle and syringe, e.g., needle pain, the risk of introducing infection to treated individuals, the risk of contamination or infection of health care workers caused by accidental needle-sticks and the disposal of used needles. In addition, such delivery affords a high degree of control over blood concentrations of administered drugs.

Recently, a novel pain-free transdermal drug delivery system that entails the use of a needleless syringe to fire solid drug-containing particles in controlled doses into and through intact skin has been described. In particular, commonly owned U.S. Pat. No. 5,630,796 to Bellhouse et al., describes a needleless syringe that delivers in a pain-free manner a pharmaceutical particles entrained in a supersonic gas flow. The needleless syringe (also referred to as “the PowderJect needleless syringe device”) is used for transdermal delivery of powdered drug compounds and compositions, for delivery of genetic material into living cells (e.g., gene therapy) and for the delivery of biopharmaceuticals to skin, muscle, blood, or lymph, as well as into and/or across mucosal surfaces. The needleless syringe can also be used in conjunction with surgery to deliver drugs and biologics to organ surfaces, solid tumors and/or to surgical cavities (e.g., tumor beds or cavities after tumor resection). Pharmaceutical agents that can be suitably prepared in a substantially solid, particulate form can be safely and easily delivered using such a device.

One particular needleless syringe generally comprises an elongate tubular nozzle having a rupturable membrane initially closing the passage through the nozzle and arranged substantially adjacent to the upstream end of the nozzle. Particles of a therapeutic agent to be delivered are disposed adjacent to the rupturable membrane and are delivered using an energizing means which applies a gaseous pressure to the upstream side of the membrane sufficient to burst the membrane and produce a supersonic gas flow (entraining the pharmaceutical particles) through the nozzle for delivery from the downstream end thereof. The particles can thus be delivered from the needleless syringe at delivery velocities as high as between Mach 1 and Mach 8 which are readily obtainable upon the bursting of the rupturable membrane.

Another needleless syringe configuration generally includes the same elements as described above, except that instead of having the pharmaceutical particles entrained within a supersonic gas flow, the downstream end of the nozzle is provided with a diaphragm that is moveable between a resting “inverted” position (in which the diaphragm presents a concavity on the downstream face to contain the pharmaceutical particles) and a discharge “everted” position (in which the diaphragm is outwardly convex on the downstream face as a result of a supersonic shockwave having been applied to the upstream face of the diaphragm). In this manner, the pharmaceutical particles contained within the concavity of the diaphragm are expelled at a high velocity from the device for transdermal delivery thereof to a targeted tissue surface, i.e., skin or mucosal surface.

Transdermal delivery using the above-described needleless syringe configurations is carried out with particles having an approximate size that generally ranges between 0.1 and 250 μm. Particles larger than about 250 μm can also be delivered from the device, with the upper limitation being the point at which the size of the particles would cause untoward damage to the skin cells. The actual distance which the delivered particles will penetrate depends upon particle size (e.g., the nominal particle diameter assuming a roughly spherical particle geometry), particle density, the initial velocity at which the particle impacts the skin surface, and the density and kinematic viscosity of the skin. Target particle densities for use in needleless injection generally range between about 0.1 and 25 g/cm³, and injection velocities generally range between about 200 and 3,000 m/sec.

A particularly unique feature of the needleless syringe is the ability to optimize the depth of penetration of delivered particles, thereby allowing for targeted administration of pharmaceuticals to various sites. For example, particle characteristics and/or device operating parameters can be selected to provide for varying penetration depths for, e.g., epidermal or dermal delivery. One approach entails the selection of particle size, particle density and initial velocity to provide a momentum density (e.g., particle momentum divided by particle frontal area) of between about 2 and 10 kg/sec/m, and more preferably between about 4 and 7 kg/sec/m. Such control over momentum density allows for optimized, tissue-selective delivery of the pharmaceutical particles.

The above-described systems provide a unique means for delivering vaccine antigens into or across skin or tissue. However, a dose-mass limitation exists for administration of powdered drug formulations using conveniently sized needleless injection devices. Thus, the dose of drug delivered can be limited thereby resulting in a residual powdered drug formulation that does not penetrate the skin or mucosal surface.

Accordingly, there is a continued need for effective and safe delivery methods of powdered therapeutic agent formulations for enhancing the dose of drug delivered.

### SUMMARY OF THE INVENTION

The present invention provides a unique method for enhancing the dose of a therapeutic agent delivered by transdermal administration.

In one embodiment, a method is provided for administering a therapeutic agent to a predetermined area of skin or mucosa of a vertebrate subject. The method comprises accelerating particles into, across or both into and across the area of skin or mucosa. Subsequently, a transdermal delivery device or an occlusive dressing is topically positioned over the area of skin mucosa. The particles, transdermal delivery device and/or the occlusive dressing contain the therapeutic agent. In addition, the method can comprise topically positioning a transdermal delivery device or an occlusive dressing over the predetermined area of skin or mucosa prior to accelerating particles into, across or both into and across the area of skin or mucosa.

In another embodiment, a method is provided for enhancing transdermal delivery of a therapeutic agent by needleless injection. The method comprises co-administering a particle comprising the therapeutic agent and a placebo particle.

These and other embodiments of the invention will readily occur to those of ordinary skill in the art in view of the disclosure herein.

### BRIEF DESCRIPTION OF THE DRAWINGS

FIG. 1 compares plasma insulin levels obtained by subcutaneous administration (squares), particle injection of a particulate insulin composition to the dermis (circles), and particle injection of a particulate insulin composition to the dermis followed by occlusion (triangles), as described in the examples.

FIG. 2 shows antibody responses in pigs injected with Hepatitis B vaccine intramuscularly (black bars), intramuscularly followed by two boosts with powdered HbsAg using the PowderJect needleless syringe (gray bars), and intramuscularly followed by two boosts with powdered HbsAg using the PowderJect needleless syringe, each boost followed by occlusion treatment (white bars), as described in the examples.

### DETAILED DESCRIPTION OF THE INVENTION

Before describing the present invention in detail, it is to be understood that this invention is not limited to particular transdermal drug delivery device configurations, particular drug/vehicle formulations, or the like, as such may vary. It is also to be understood that the terminology used herein is for the purpose of describing particular embodiments only, and is not intended to be limiting.

It must be noted that, as used in this specification and the appended claims, the singular forms “a,” “an” and “the” include plural referents unless the content clearly dictates otherwise. Thus, for example, reference to “a permeation enhancer” includes a mixture of two or more permeation enhancers, reference to “an excipient” or “a vehicle” includes mixtures of excipients or vehicles, reference to “a particle” includes reference to two or more such particles, and the like.

Unless defined otherwise, all technical and scientific terms used herein have the same meaning as commonly understood by one of ordinary skill in the art to which the invention pertains. Although any methods and materials similar or equivalent to those described herein can be used in the practice for testing of the present invention, the preferred materials and methods are described herein.

In describing and claiming the present invention, the following terminology will be used in accordance with the definitions set out below.

As used herein, the terms “pharmaceutical agent,” “pharmaceutically active agent,” “therapeutic agent” or “therapeutically active agent” are used interchangeably and intend any compound or composition of matter which, when administered to an organism (human or animal) induces a desired pharmacologic and/or physiologic effect by local and/or systemic action. The term therefore encompasses those compounds or chemicals traditionally regarded as drugs and vaccines, as well as biopharmaceuticals including molecules such as peptides, hormones, nucleic acids, gene constructs and the like.

The term “transdermal” delivery captures both transdermal (or “percutaneous”) and transmucosal administration, i.e., delivery by passage of a therapeutic agent into or through the skin or mucosal tissue. See, e.g., Transdermal Drug Delivery: Developmental Issues and Research Initiatives, Hadgraft and Guy (eds.), Marcel Dekker, Inc., (1989); Controlled Drug Delivery: Fundamentals and Applications, Robinson and Lee (eds.), Marcel Dekker Inc., (1987); and Transdermal Delivery of Drugs, Vols. 1-3, Kydonieus and Berner (eds.), CRC Press, (1987). Aspects of the invention which are described herein in the context of “transdemnal” delivery, unless otherwise specified, are meant to apply to both transdermal and transmucosal delivery. That is, the compositions, systems, and methods of the invention, unless explicitly stated otherwise, should be presumed to be equally applicable to transdermal and transmucosal modes of delivery.

The term “passive transdermal delivery” refers to the delivery into or through a body surface (e.g., skin) of a pharmaceutically acceptable composition without the aid of an applied electromotive force. Passive transdermal delivery can be accomplished using a number of means including, without limitation, direct application to the skin, transdermal patches, membrane-moderated systems to provide controlled delivery, adhesive diffusion-controlled systems, matrix dispersion-type systems, and microreservoir systems. Such systems are known in the art and are discussed in detail in Remington: The Science and Practice of Pharmacy, Mack Publishing Company, Easton, Pa., 19th edition, 1995.

The terms “electrotransport,” “iontophoresis,” and “iontophoretic” are used herein to refer to the delivery into or through a body surface (e.g., skin) of one or more pharmaceutically acceptable compositions by means of an applied electromotive force to a composition-containing reservoir. Such delivery device is alternatively referred to herein as an “active transdermal delivery device.” The agent may be delivered by electromigration, electroporation, electroosmosis or any combination thereof. Electroosmosis has also been referred to as electrohydrokinesis, electro-convection, and electrically induced osmosis. In general, electroosmosis of a species into a tissue results from the migration of solvent in which the species is contained, as a result of the application of electromotive force to the therapeutic species reservoir, i.e., solvent flow induced by electromigration of other ionic species. During the electrotransport process, certain modifications or alterations of the skin may occur such as the formation of transiently existing pores in the skin, also referred to as “electroporation.” Any electrically assisted transport of species enhanced by modifications or alterations to the body surface (e.g., formation of pores in the skin) are also included in the term “electrotransport” as used herein. Thus, as used herein, the terms “electrotransport,” “iontophoresis” and “iontophoretic” refer to (1) the delivery of charged agents by electromigration, (2) the delivery of uncharged agents by the process of electroosmosis, (3) the delivery of charged or uncharged agents by electroporation, (4) the delivery of charged agents by the combined processes of electromigration and electroosmosis, and/or (5) the delivery of a mixture of charged and uncharged agents by the combined processes of electromigration and electroosmosis.

By “needleless syringe” is meant an instrument which delivers a particulate composition transdermally, without a conventional needle that pierces the skin. Needleless syringes for use with the present invention are discussed throughout this document.

An “occlusive dressing” is one that, when applied to a predetermined area of skin or mucosa, alters environmental aspects of the area of skin or mucosa that facilitate transdermal delivery of pharmaceutically acceptable compositions. An occlusive dressing can provide a physical barrier to prevent the escape, or enhance the delivery, of a material applied to and residing at or near the surface of the area of skin or mucosa. In addition, application of an occlusive dressing can result in an increase or can prevent the decrease in the moisture content, pH, oxygen environment, or the like, of the area of skin or mucosa. As used herein, the term “occlusive dressing” is intended to include a bandage, a gas- and/or moisture-permeable or impermeable synthetic polymer-based dressing, or other solid-form dressing prepared from natural or synthetic materials or a combination thereof, a topical formulation, such as a cream, a gel, an ointment, or other liquid or semi-liquid material, and the like. An occlusive dressing can be prepared to contain a locally or systemically active therapeutic agent.

By an “effective” amount of a therapeutic agent is meant a nontoxic but sufficient amount of the agent to provide the desired therapeutic or prophylactic effect. An “effective” amount of a permeation enhancer as used herein means an amount that will provide the desired increase in skin or mucosa permeability and, correspondingly, the desired depth of penetration, rate of administration, and amount of therapeutic agent delivered.

By “antigen” is meant a molecule that contains one or more epitopes that will stimulate a host's immune system to make a cellular antigen-specific immune response, or a humoral antibody response. Thus, antigens include proteins, polypeptides, antigenic protein fragments, oligosaccharides, polysaccharides, and the like. Furthermore, the antigen can be derived from any known virus, bacterium, parasite, plants, protozoans, or fungus, and can be a whole organism or immunogenic parts thereof, e.g., cell wall components. The term also includes tumor antigens. Similarly, an oligonucleotide or polynucleotide which expresses an antigen, such as in DNA immunization applications, is also included in the definition of antigen. Synthetic antigens are also included in the definition of antigen, for example, haptens, polyepitopes, flanking epitopes, and other recombinant or synthetically derived antigens (Bergmann et al. (1993) Eur. J. Immunol. 23:2777-2781; Bergmann et al. (1996) J. Immunol. 157:3242-3249; Suhrbier, A. (1997) Immunol. and Cell Biol. 75:402-408; Gardner et al. (1998) 12th World AIDS Conference, Geneva, Switzerland, Jun. 28-Jul. 3, 1998).

The term “vaccine composition” intends any pharmaceutical composition containing an antigen, which composition can be used to prevent or treat a disease or condition in a subject. The term thus encompasses both subunit vaccines, i.e., vaccine compositions containing antigens which are separate and discrete from a whole organism with which the antigen is associated in nature, as well as compositions containing whole killed, attenuated or inactivated bacteria, viruses, parasites or other microbes.

Viral vaccine compositions used herein include, but are not limited to, those containing, or derived from, members of the families Picornaviridac (e.g., polioviruses, etc.); Caliciviridae; Togaviridae (e.g., rubella virus, dengue virus, etc.); Flaviviridae; Coronaviridae; Reoviridae; Birnaviridae; Rhabodoviridae (e.g., rabies virus, etc.); Filoviridae; Paramyxoviridae (e.g., mumps virus, measles virus, respiratory syncytial virus, etc.); Orthomyxoviridae (e.g., influenza virus types A, B and C, etc.); Bunyaviridae; Arenaviridae; Retroviradae (e.g., HTLV-I; HTLV-II; HIV-1; and HIV-2); simian immunodeficiency virus (SIV) among others.

Additionally, viral antigens may be derived from papillomavirus (e.g., HPV); a herpesvirus; a hepatitis virus, e.g., hepatitis A virus (HAV), hepatitis B virus (HBV), hepatitis C virus (HCV), the delta hepatitis virus (HDV), hepatitis E virus (HEV) and hepatitis G virus (HGV); and the tick-borne encephalitis viruses. See, e.g. Virology, 3rd Edition (W. K. Joklik ed. 1988); Fundamental Virology, 2nd Edition (B. N. Fields and D. M. Knipe, eds. 1991), for a description of these and other viruses. Bacterial vaccine compositions used herein include, but are not limited to, those containing or derived from organisms that cause diphtheria, cholera, tuberculosis, tetanus, pertussis, meningitis, and other pathogenic states, including, Meningococcus A, B and C, Hemophilus influenza type B (HIB), and Helicobacter pylori. Examples of anti-parasitic vaccine compositions include those derived from organisms causing malaria and Lyme disease.

As used herein, the term “treatment” includes any of following: the prevention of infection or reinfection; the reduction or elimination of symptoms; and the reduction or complete elimination of a pathogen. Treatment may be effected prophylactically (prior to infection) or therapeutically (following infection).

By “vertebrate subject” is meant any member of the subphylum cordata, particularly mammals, including, without limitation, humans and other primates. The term does not denote a particular age. Thus, both adult and newborn individuals are intended to be covered.

The phrase “predetermined area of skin” is intended to be a defined area of intact unbroken living skin or mucosal tissue. That area will usually be in the range of about 0.3 cm² to about 30 cm², more usually in the range of about 5 cm² to about 10 cm². However, it will be appreciated by those skilled in the art of transdermal drug delivery that the area of skin or mucosal tissue through which drug is administered may vary significantly, depending on device configuration, dose, and the like.

“Penetration enhancement” or “permeation enhancement” as used herein relates to an increase in the permeability of skin to a therapeutic agent, i.e., so as to increase the rate at which the agent permeates through the skin and enters the bloodstream. The enhanced permeation effected through the use of such enhancers can be observed by measuring the rate of diffusion of agent through animal or human skin using a diffusion cell apparatus well known in the art. Penetration enhancers can be used to facilitate absorption into or through the skin. Such penetration enhancers include solvents such as water, alcohols including methanol, ethanol, 2-propanol and the like, ethyl glycerol, methyl nicotinate, alkyl methyl sulfoxides, pyrrolidones, laurocapram, acetone, dimethylacetamide, dimethyl formamide, tetrahydrofurfuryl; surfactants; glycerol monoesters (GMOs), e.g., glycerol monooleate; fatty acids; fatty acid esters; terpenes; and chemicals such as urea, N,N-diethyl-m-toluamide, and the like.

“Carriers” or “vehicles” as used herein refer to carrier materials suitable for transdermal drug administration, and include any such materials known in the art, e.g., any liquid, gel, solvent, liquid diluent, solubilizer, or the like, which is nontoxic and which does not interact with other components of the composition in a deleterious manner. Examples of suitable carriers for use herein include water, silicone, liquid sugars, waxes, petroleum jelly, and a variety of other materials. The term “carrier” or “vehicle” as used herein may also refer to stabilizers, crystallization inhibitors, or other types of additives useful for facilitating transdermal drug delivery.

“Optional” or “optionally” means that the subsequently described event or circumstance may or may not occur, and that the description includes instances where said event or circumstance occurs and instances where it does not. For example, the phrase “optionally followed by application of a transdermal delivery device” means that application of such a device may or may not be occur and that the description includes both when application of such a device occurs and when application of such a device does not occur.

The invention is directed to a method by which flux of a therapeutic agent painlessly administered to the skin or mucosa of a subject using a needleless syringe is enhanced by the subsequent application of a transdermal delivery device or an occlusive dressing, or both a transdermal delivery device and an occlusive dressing to the site of agent administration. In addition, the method may result in a reduction of some side effects and/or local skin reactions.

The method comprises accelerating particles into, across or both into and across the area of skin or mucosa. A transdermal delivery device or occlusive dressing is then topically positioned over the area of skin or mucosa. In one alternate embodiment, the method comprises a pretreatment step of accelerating placebo particles, i.e., particles that contain no therapeutic agent; particles that contain an adjuvant; or particles that contain an agent, e.g., a vasoactive agent or a permeation enhancing agent, into the skin or mucosa. This pretreatment that is intended to prepare the predetermined area of skin or mucosa for the subsequent administration of a therapeutic agent by application of a transdermal delivery device or occlusive dressing containing such an agent. In another alternative embodiment, the method comprises first pretreating the preselected area of skin or mucosa with a transdermal drug delivery device or occlusive dressing, optionally containing a permeation enhancing agent, vasoactive agent, or the like, to prepare the skin or mucosa for the subsequent treatment. The area of skin or mucosa is then treated by accelerating particles into and/or across the area of skin or mucosa and, subsequently, applying to the area of skin a transdermal delivery device or occlusive dressing.

In a further alternative embodiment, both particles containing a therapeutic agent and placebo particles are sequentially or simultaneously administered to the area of skin or mucosa. Preferably, the particles are accelerated using a needleless syringe as described more fully hereinbelow. Co-administration of active agent and placebo particles is, optionally, followed by application of a transdermal delivery device or occlusive dressing the area of skin or mucosa.

When present in the formulation to be delivered, the placebo particles can comprise inert dense particles, such as gold, tungsten, metal particles coated with a permeation enhancing agent or surfactant, or the like, that are up to 75 μm in diameter, preferably about 1 μm to 50 μm in diameter and included in the range of about 0.1% to about 10% of the total mass of particles in the formulation. Pretreatment of an area of skin or mucosa with placebo particles increases the rate at which transdermal drug delivery from the transdermal delivery device or occlusive dressing is achieved.

After the pretreatment of the area of skin or mucosa with the particles, a transdermal delivery device and/or an occlusive dressing is positioned topically oil the pretreated area of skin or mucosa. Application of the device or dressing is intended to occur either concurrently or immediately after administration of the particles, for at least the goal of protecting residual particle powder remaining on the surface of the skin or mucosa from accidental or intentional abrasive removal and, in addition, for the goal of altering the local environment of the area of skin or mucosa or increasing the amount particle powder available for continued transdermal flux and reducing local skin reactions to enhance recovery. However, under circumstances in which abrasive removal of residual powder is not considered an immediate concern, e.g., when the particles are administered to an otherwise protected or inaccessible area of skin or mucosa, application of the transdermal delivery device or occlusive dressing can be delayed.

The transdermal delivery device can be a passive transdermal drug delivery device, an active drug delivery device, e.g., an electrotransport drug delivery device, or the like. The occlusive dressing can be a bandage, or like material, or an occlusive topical formulation. Optionally, the transdermal delivery device or occlusive dressing contains a therapeutic agent.

In one embodiment, the needleless syringe is used to administer a desired therapeutic agent to the predetermined area of skin or mucosa, which may result in a residue of particle powder on the surface of the skin or mucosa. The administration by needleless injection is followed by application to the area of skin or mucosa of a transdermal delivery device or occlusive dressing that contains no therapeutic agent and acts to prevent residual drug loss to the environment. In this case, the transdermal delivery device or occlusive dressing may contain no active agents, an anti-irritant such as a topical corticoid, glycerine/green tea, and the like, a permeation enhancing agent, a vasoactive agent, or the like, that acts to enhance uptake of the active agent administered by the needleless syringe.

In a second embodiment, the administration of a therapeutic agent by needleless syringe to the predetermined area of skin or mucosa is followed by the application of a transdermal delivery device or occlusive dressing that contains a therapeutic agent. The therapeutic agent administered from the transdermal delivery device or occlusive dressing may be the same or different as that administered by needleless syringe.

The method of the invention can be used to administer an immunizing composition that contains, in combination, a selected antigen and an adjuvant. In one embodiment, the method comprises optionally applying to a predetermined area of skin or mucosa of a vertebrate subject a transdermal delivery device or an occlusive dressing comprising an adjuvant and, after a period of time ranging from 1 minute to 24 hours or longer, removing the device or dressing and administering by needleless injection to the area of skin or mucosa particles comprising a selected antigen and, optionally, the same or a different adjuvant. Subsequently, a transdermal drug delivery device or an occlusive dressing can be topically positioned over the area of skin mucosa to enhance uptake of the composition. The final device or dressing optionally contains an adjuvant.

In yet another embodiment, the needleless syringe may be used to administer to a predetermined area of skin or mucosa a placebo composition that contains no therapeutic agent, followed by application thereto of a transdermal delivery device or occlusive dressing containing a therapeutic agent. While not wishing to be bound by theory, the inventors have shown that, in this embodiment, pretreatment of the area of skin or mucosa with the needleless syringe acts to enhance the permeability or reduce the penetration barriers of the area of skin or mucosa, thereby increasing the transdermal delivery of therapeutic agent to the subject from the transdermal delivery device or occlusive dressing. The delivery of larger therapeutically active biomolecules, such as polypeptides, can also be more efficiently effected following treatment of the area of skin or mucosa with the needleless syringe. Treatment of the skin or mucosa with a placebo composition will also allow the administration of an active agent using a transdermal delivery device or occlusive dressing having a smaller skin- or mucosa-contact surface area.

The method can also be used to modify or design the pharmacokinetic profile of therapeutic agent delivery. By “pharmacokinetic profile” is intended the variation with time of agent concentration in, for example, skin, muscle, lymph, blood, serum or plasma, as a result of absorption, distribution and elimination of the agent. A graphical representation of, e.g., plasma therapeutic agent concentration versus time will be indicative of the effective concentration of a single dose of the agent characterized by its latency, time of peak effect, magnitude of peak effect and duration of effect. These characteristics can be affected by alterations in the rate of absorption, dose of therapeutic agent administered and rate of elimination.

Thus, for example, administration to a predetermined area of skin or mucosa of a therapeutic agent by needleless syringe can provide a bolus of the agent that results in a rapidly developing peak of agent concentration in blood, etc., followed by a relatively rapid decline in agent concentration. This pharmacokinetic profile can be modified, e.g., by the subsequent application of a transdermal delivery device or occlusive dressing that, optionally, contains the same therapeutic agent to the area of skin or mucosa, to extend the duration of effective blood concentration of the agent.

A typical pharmacokinetic profile for a transdermal delivery of a therapeutic agent to a predetermined area of skin or mucosa is a prolonged absorption period followed by a relatively long-lived duration of effective agent concentration in the blood. The pharmacokinetic profile can be modified by enhancing the absorption phase of therapeutic agent administration by pretreatment of the predetermined area of skin or mucosa with, for example, a needleless injection of placebo particles or placebo particles coated with a permeation enhancer. Such pretreatment can reduce the lag time in achieving therapeutic levels of the subsequently delivered agent.

Based on the foregoing disclosure, one of ordinary skill in the art will be able use needleless syringe technology in combination with a transdermal delivery device and/or occlusive dressing to design dosing regimens to effect any desired pharmacokinetic profile.

One particularly preferred needleless syringe useful in the method disclosed and claimed herein is described in commonly owned U.S. Pat. No. 5,630,796 (“the '796 patent”). The syringe is used for transdermal delivery of powdered therapeutic compounds and compositions to skin, muscle, blood or lymph. The syringe can also be used in conjunction with surgery to deliver therapeutics to organ surfaces, solid tumors and/or to surgical cavities (e.g., tumor beds or cavities after tumor resection).

Additional embodiments of the above-described syringe are disclosed in the '796 patent commonly owned U.S. application Ser. No. 08/800,016 for “PARTICLE DELIVERY,” by Bellhouse et al., filed Feb. 13, 1997.

A second preferred embodiment of a needleless syringe, disclosed in commonly owned U.S. application Ser. No. 08/860,403. the disclosure of which is incorporated herein by reference, is provided having a body containing a lumen. An upstream end of the lumen is, or is arranged to be, connected to a source of gaseous pressure which can be suddenly released into the lumen. The downstream end of the lumen terminates behind an eversible diaphragm which is movable between an inverted position which provides a concavity for containing particles comprising a therapeutic agent, and an everted, outwardly convex, position. The eversible diaphragm is arranged such that, when an energizing gas flow is released into the lumen, the diaphragm will travel from its inverted to its everted position, thereby projecting the particles from the diaphragm toward a target surface.

One type of transdermal delivery device that finds utility in the method disclosed and claimed herein is a passive transdermal system as described in U.S. Pat. No. 4,379,454 to Campbell et al. the disclosure of which is incorporated herein by reference. The system is a laminate comprising a backing layer, a drug reservoir layer containing drug, a contact adhesive and a release liner. Optionally, a rate-controlling membrane is interposed between drug reservoir layer and contact adhesive. In another embodiment, the contact adhesive layer may serve also as the drug reservoir layer.

The backing layer functions as the primary structural element of the device and provides the device with much of its flexibility, drape and, preferably, occlusivity. The material used for the backing layer should be inert and incapable of absorbing drug, enhancer or other components of the pharmaceutical composition contained within the device. The backing is preferably made of one or more sheets or films of a flexible elastomeric material that serves as a protective covering to prevent loss of drug and/or vehicle via transmission through the upper surface of the device, and will preferably impart a degree of occlusivity to the device, such that the area of the skin or mucosa covered on application becomes and/or remains hydrated. The material used for the backing layer should permit the device to follow the contours of the skin and be worn comfortably on areas of skin such as at joints or other points of flexure, that are normally subjected to mechanical strain with little or no likelihood of the device disengaging from the skin due to differences in the flexibility or resiliency of the skin and the device. Examples of materials useful for the backing layer are polyesters, polyethylene, polypropylene, polyurethanes and polyether amides. The layer is preferably in the range of about 15 microns to about 250 microns in thickness, and may, if desired, be pigmented, metallized, or provided with a matte finish suitable for writing.

The drug reservoir layer provides a means for containing drug. As mentioned above, the drug reservoir may also comprise the contact adhesive. The reservoir layer will generally range in thickness from about 1 to about 100 microns, preferably approximately 25 to 75 microns.

A pharmaceutically acceptable contact adhesive layer functions to secure the device to the skin during use. In an alternate embodiment, a peripheral ring of contact adhesive is provided on the basal surface of the device. In an additional alternate embodiment, the adhesive layer may also serve as the drug reservoir layer. Suitable contact adhesive materials are pressure-sensitive adhesives suitable for long-term skin contact, which are also physically and chemically compatible with the drug formulations, i.e., the drug itself and any carriers and vehicles employed. Preferred materials for this layer include, for example, polysiloxanes, polyisobutylenes, polyacrylates, polyurethanes, plasticized ethylene-vinyl acetate copolymers, low molecular weight polyether amide block polymers (e.g., PEBAX), tacky rubbers such as polyisobutene, polystyrene-isoprene copolymers, polystyrene-butadiene copolymers, and mixtures thereof.

Additionally, to protect the basal surface of the device during storage and just prior to use, a release liner is provided to cover the adhesive layer. The release liner is a disposable element that serves only to protect the device prior to application. Typically, the release liner is formed from a material impermeable to the drug, vehicle and adhesive, and which is easily stripped from the contact adhesive. Release liners are typically treated with silicone or fluorocarbons. Silicone-coated polyester is presently preferred.

It may also be desirable to include a rate-controlling membrane in between the drug reservoir and a contact adhesive layer, when present. The materials used to form such a membrane are selected to limit the flux of non-drug components, i.e., enhancers, vehicles, and the like, from the drug reservoir, while not limiting the flux of drug. Representative materials useful for forming rate-controlling membranes include polyolefins such as polyethylene and polypropylene, polyamides, polyesters, ethylene-ethacrylate copolymer, ethylene-vinyl acetate copolymer, ethylene-vinyl methylacetate copolymer, ethylene-vinyl ethylacetate copolymer, ethylene-vinyl propylacetate copolymer, polyisoprene, polyacrylonitrile, ethylene-propylene copolymer, and the like. A particularly preferred material useful to form the rate controlling membrane is ethylene-vinyl acetate copolymer.

It will be appreciated by those working in the field that the present invention can be used in conjunction with a wide variety of passive transdermal systems, as the invention is not limited in this regard. For examples of passive systems, reference may be had to, but not limited to, U.S. Pat. No. 4,588,580 to Gale et al., U.S. Pat. No. 4,832,953 to Campbell et al., U.S. Pat. No. 4,698,062 to Gale et al., U.S. Pat. No. 4,867,982 to Campbell et al., and U.S. Pat. No. 5,268,209 to Hunt at al., of which any of the disclosed systems can be used with the present invention.

A transdermal system as described above may be prepared as follows. An adhesive is cast onto a release liner. Solvent is evaporated therefrom, and the adhesive is then laminated onto the drug reservoir, which is in turn transfer-laminated onto the backing film. Alternatively, the drug reservoir may first be laminated to the backing layer and subsequently laminated to the precast adhesive layer.

It will be appreciated by those working in the field that the invention can also be used in conjunction with a wide variety of electrotransport drug delivery systems, as the method is not limited in any way in this regard. For examples of electrotransport drug delivery systems, reference may be had to U.S. Pat. No. 5,147,296 to Theeuwes et al., U.S. Pat. No. 5,080,646 to Theeuwes et al., U.S. Pat. No. 5,169,382 to Theeuwes et al., and U.S. Pat. No. 5,169,383 to Gyory et al., as well as to U.S. Pat. Nos. 5,224,927, 5,224,928, 5,246,418, 5,320,597, 5,358,483 and 5,135,479, and UK Patent Application No. 2 239 803, the disclosures of which are incorporated herein by reference

As described in these patent documents, a representative electrotransport delivery device that may be used in conjunction with the present method comprises an upper housing, a circuit board assembly and associated electronic circuitry, a lower housing, an anode electrode, a cathode electrode, an anode drug/chemical reservoir, a cathode drug/chemical reservoir, a skin-compatible adhesive and a battery, all of which are integrated into a self-contained unit. The anode and cathode electrodes are in direct mechanical and electrical with the top sides of the reservoirs. The bottom sides of the reservoirs contact the patient's skin through openings in the adhesive, by which means the device adheres to the patient's body.

The anodic electrode is comprised of a metal species that is capable of undergoing oxidation during operation of the device, e.g., silver, and the cathodic electrode is preferably comprised of a chemical species capable of undergoing reduction during operation of the device, e.g., silver chloride.

The reservoirs generally comprise a gel matrix, with the drug solution uniformly dispersed in at least one of the reservoirs. Drug concentrations in the range of approximately 1×10⁻⁴ M to 1.0 M or more can be used, with drug concentrations in the lower portion of the range being preferred. Suitable polymers for the gel matrix may comprise essentially any nonionic synthetic and/or naturally occurring polymeric materials. A polar nature is preferred when the active agent is polar and/or capable of ionization, so as to enhance agent solubility. Optionally, the gel matrix will be water swellable. Examples of suitable synthetic polymers include, but are not limited to, poly(acrylamide), poly(2-hydroxyethyl acrylate), poly(2-hydroxypropyl acrylate), poly(N-vinyl-2-pyrrolidone), poly(n-methylol acrylamide), poly(diacetone acrylamide), poly(2-hydroxylethyl methacrylate), poly(vinyl alcohol) and poly(allyl alcohol). Hydroxyl functional condensation polymers (i.e., polyesters, polycarbonates, polyurethanes) are also examples of suitable polar synthetic polymers. Polar naturally occurring polymers (or derivatives thereof) suitable for use as the gel matrix are exemplified by cellulose ethers, methyl cellulose ethers, cellulose and hydroxylated cellulose, methyl cellulose and hydroxylated methyl cellulose, gums such as guar, locust, karaya, xanthan, gelatin, and derivatives thereof. Ionic polymers can also be used for the matrix provided that the available counterions are either drug ions or other ions that arc oppositely charged relative to the active agent.

When electrical current flows through the device, oxidation of a metal species or reduction of a chemical species takes place along the surface of at least one of the anodic or cathodic electrodes. Although a variety of electrochemical reactions can be utilized, a preferred reaction is a class of charge transfer reactions whereby a portion of at least one of the anodic or cathodic electrodes participates in a charge transferring chemical reaction, i.e., a material in at least one of the anodic or cathodic electrodes is consumed or generated. This is accomplished through oxidation and/or reduction reactions occurring at the electrodes. Examples of preferred oxidation/reduction reactions include the following:
Ag⇄Ag⁺+e⁻;
Zn⇄Zn²⁺+2e⁻;
Cu⇄Cu²⁺+2e⁻;
Ag+Cl⁻⇄AgCl+e⁻; and
Zn+2Cl⇄ZnCl₂+2e⁻
where the forward reaction is the oxidation reaction taking place at the anodic electrode and the reverse reaction is the reduction reaction taking place at the cathodic electrode. Other standard electrochemical reactions and their respective reduction potentials are well known in the art. See CRC Handbook of Chemistry and Physics, pp. 8-21 to 8-31, 75th edn. (1994-1995).

Any number of therapeutic agents and/or particles of therapeutic agents can be administered to an organism by needleless syringe to induce a desired pharmacologic, immunogenic, and/or physiologic effect by local and/or systemic action. Such therapeutic agents include those compounds or chemicals traditionally regarded as drugs, vaccines, and biopharmaceuticals including molecules such as proteins, peptides, hormones, nucleic acids, gene constructs and the like. In addition, compounds or compositions may be administered by needleless syringe for use in all of the major therapeutic areas including, but not limited to, anti-infectives such as antibiotics and antiviral agents; analgesics and analgesic combinations; local and general anesthetics; anorexics; antiarthritics; antiasthmatic agents; anticonvulsants; antidepressants; antihistamines; anti-inflammatory agents; antinauseants; antimigrane agents; antineoplastics; antipruritics; antipsychotics; antipyretics; antispasmodics; cardiovascular preparations (including calcium channel blockers, beta-blockers, beta-agonists and antiarrythmics); antihypertensives; diuretics; vasodilators; central nervous system stimulants; cough and cold preparations; decongestants; diagnostics; hormones; bone growth stimulants and bone resorption inhibitors; immunosuppressives; muscle relaxants; psychostimulants; sedatives; tranquilizers; proteins, peptides, and fragments thereof (whether naturally occurring, chemically synthesized or recombinantly produced); and nucleic acid molecules (polymeric forms of two or more nucleotides, either ribonucleotides (RNA) or deoxyribonucleotides (DNA) including double- and single-stranded molecules and supercoiled or condensed molecules, gene constructs, expression vectors, plasmids, antisense molecules and the like).

Particles of a therapeutic agent, alone or in combination with another drug or agent, are typically prepared as pharmaceutical compositions which can contain one or more added materials such as carriers, vehicles, and/or excipients. “Carriers,” “vehicles” and “excipients” generally refer to substantially inert materials that are nontoxic and do not inter-act with other components of the composition in a deleterious manner. These materials can be used to increase the amount of solids in particulate pharmaceutical compositions. Examples of suitable carriers include silicone, gelatin, waxes, and like materials. Examples of normally employed “excipients,” include pharmaceutical grades of dextrose, sucrose, lactose, trehalose, mannitol, sorbitol, inositol, dextran, starch, cellulose, sodium or calcium phosphates, calcium sulfate, citric acid, tartaric acid, glycine, high molecular weight polyethylene glycols (PEG), erodible polymers (such as polylactic acid, polyglycolic acid, and copolymers thereof), and combinations thereof. In addition, it may be desirable to include a charged lipid and/or detergent in the pharmaceutical compositions. Such materials can be used as stabilizers, anti-oxidants, or used to reduce the possibility of local irritation at the site of administration. Suitable charged lipids include, without limitation, phosphatidylcholines (lecithin), and the like. Detergents will typically be a nonionic, anionic, cationic or amphoteric surfactant. Examples of suitable surfactants include, for example, Tergitol®and Triton® surfactants (Union Carbide Chemicals and Plastics, Danbury, Conn.), polyoxyethylenesorbitans, e.g., TWEEN® surfactants (Atlas Chemical Industries, Wilmington, Del.), polyoxyethylene ethers, e.g., Brij, pharmaceutically acceptable fatty acid esters, e.g., lauryl sulfate and salts thereof (SDS), and like materials.

The needleless syringes can be used for transdermal delivery of powdered therapeutic agents and compositions, for delivery of genetic material into living cells (e.g., gene therapy or nucleic acid vaccination), both in vivo and ex vivo, and for the delivery of biopharmaceuticals to skin, muscle, blood or lymph. The syringes can also be used in conjunction with surgery to deliver therapeutic agents, drugs, immunogens, and/or biologics to organ surfaces, solid tumors and/or to surgical cavities (e.g., tumor beds or cavities after tumor resection). In theory, practically any agent that can be prepared in a substantially solid, particulate form can be safely and easily delivered using the present devices.

Delivery of therapeutic agents from a needleless syringe system is generally effected using particles having an approximate size generally ranging from 0.1 to 250 μm. For drug delivery, the optimal particle size is usually at least about 10 to 15 μm (the size of a typical cell). For gene delivery, the optimal particle size is generally substantially smaller than 10 μm. Particles larger than about 250 μm can also be delivered from the devices, with the upper limitation being the point at which the size of the particles would cause untoward damage to the skin cells. The actual distance which the delivered particles will penetrate a target surface depends upon particle size (e.g., the nominal particle diameter assuming a roughly spherical particle geometry), particle density, the initial velocity at which the particle impacts the surface, and the density and kinematic viscosity of the targeted skin or mucosal tissue. In this regard, optimal particle densities for use in needleless injection generally range between about 0.1 and 25 g/cm³, preferably between about 0.9 and 1.5 g/cm³, and injection velocities can range from about 200 to about 3,000 m/sec, preferably 200 to 2,500 m/sec.

When nucleic acid preparations, e.g., DNA molecules, are to be delivered using the devices of the present invention, the preparations are optionally encapsulated, adsorbed to, or associated with, carrier particles. Suitable carrier particles can be comprised of any high density, biologically inert material. Dense materials are preferred in order to provide particles that can be readily accelerated toward a target over a short distance, wherein the particles are still sufficiently small in size relative to the cells into which they are to be delivered.

In particular, tungsten, gold, platinum and iridium carrier particles can be used. Tungsten and gold particles are preferred. Tungsten particles are readily available in average sizes of 0.5 to 2.0 μm in diameter, and are thus suited for intracellular delivery. Gold is a preferred material since it has high density, is relatively inert to biological materials and resists oxidation, and is readily available in the form of spheres having an average diameter of from about 0.2 to 3 μm.

When desired, any of a number of therapeutic agents can be incorporated into a transdermal delivery system or occlusive dressing, i.e., any compound suitable for transdermal or transmucosal administration which induces a desired systemic or local effect. Such substances include the broad classes of compounds normally delivered through body surfaces and membranes, including skin. In general, this includes: anti-infectives such as antibiotics and antiviral agents; analgesics and analgesic combinations; anorexics; antihelminthics; antiarthritics; antiasthmatic agents; anticonvulsants; antidepressants; antidiabetic agents; antidiarrheals; antihistamines; antiinflammatory agents; antimigraine preparations; antinauseants; antineoplastics; antiparkinsonism drugs; antipruritics; antipsychotics; antipyretics; antispasmodics; anticholinergics; sympathomimetics; xanthine derivatives; cardiovascular preparations including calcium channel blockers and beta-blockers such as pindolol and antiarrhythmics; antihypertensives; diuretics; vasodilators including general coronary, peripheral and cerebral; central nervous system stimulants; cough and cold preparations, including decongestants; hormones such as estradiol and other steroids, including corticosteroids; hypnotics; immunosuppressives; muscle relaxants; parasympatholytics; psychostimulants; sedatives; and tranquilizers.

Preferred therapeutic agents to be administered in conjunction with the transdermal systems of the invention are those which typically display low skin flux, e.g., steroid drugs, calcium channel blockers and potassium channel blockers. Other preferred therapeutic agents are those that require high flux to achieve a desired therapeutic effect and thus may require the presence of an enhancer in the drug formulation.

The therapeutic agent formulations may also include standard carriers or vehicles useful for facilitating drug delivery, e.g., stabilizers, antioxidants, anti-irritants and crystallization inhibitors.

Skin permeation enhancers may also be present in the transdermal delivery device or occlusive dressing drug formulation. If an enhancer is incorporated in the device, it will generally represent on the order of approximately 1 wt. % to 25 wt. % of the drug formulation. Suitable enhancers include, but are not limited to, dimethylsulfoxide (DMSO), dimethyl formamide (DMF), N,N-dimethylacetamide (DMA), N-methylpyrollidone, decylmethylsulfoxide (C₁₀MSO), polyethylene glycol monolaurate (PEGML), propylene glycol (P G), propylene glycol monolaurate (PGML), glycerol monolaurate (GML), methyl laurate (ML), lauryl lactate (LL), isopropyl myristate (IPM), terpenes such as menthone, C₂-C₆ diols, particularly 1,2-butanediol, lecithin, the 1-substituted azacycloheptan-2-ones, particularly 1-n-dodecylcyclazacycloheptan-2-one (available under the trademark Azone® from Whitby Research Incorporated, Richmond, Va.), alcohols, and the like. Vegetable oil permeation enhancers, as described in U.S. Pat. No. 5,229,130 to Sharma, may also be used. Such oils include, for example, safflower oil, cotton seed oil and corn oil. Film-forming polymer compositions and their use for delivery of pharmaceutically active agents to the skin are described in U.S. Pat. No. 5,807,957 to Samour et al.

Preferred therapeutic agent formulations, i.e., the drug-containing composition loaded into the drug reservoir, will typically contain on the order of about 0.1 wt. % to 20 wt. %, preferably about 1 wt. % to 10 wt. % drug, with the remainder of the formulation representing other components such as enhancers, vehicles or the like.

### Experimental

Below are examples of specific embodiments for carrying out the present invention. The examples are offered for illustrative purposes only, and are not intended to limit the scope of the present invention in any way. Efforts have been made to ensure accuracy with respect to numbers used (e.g., amounts, temperatures, etc.), but some experimental error and deviation should, of course, be allowed for.

### EXAMPLE 1

### Enhanced Flux of Insulin Administered using a Needleless Syringe

In order to assess the ability of an occlusive dressing to enhance the flux of insulin administered via a particle injection device, the following in vitro studies were carried out. For all studies, a modified Franz cell (6.9 ml) was used to investigate transdermal flux. The receiver medium in the Franz cell was phosphate buffered saline (PBS) adjusted to pH 9.0 with NaOH and to which was added 2.5% TWEEN® 80 to minimize insulin adsorption to the glass and to maintain infinite sink conditions. In addition, the temperature of the receiver medium was maintained at 32° C. during the studies. The top of the Franz cell was covered with a full thickness (about 300 mm) sample of human cadaver skin.

Porcine insulin (100%) was obtained from a commercial source and lyophilized, compressed and then milled to produce insulin particles suitable for use in a particle injection device. This particle formation procedure is described in our commonly-owned International Publication No. WO 97/48485, published 24 Dec. 1997, which publication is incorporated herein by reference. Different sieve fractions were obtained using standard mesh screens.

All particle injections were carried out using a PowderJect® needleless syringe such as the device described in commonly owned U.S. Pat. No. 5,630,796 (the '796 patent). As described in the '796 patent, incorporated herein by reference, particle payloads (in these studies, 1, 2 or 3 mg) were loaded into a trilaminate drug cassette having upper and lower rupturing membranes. The devices were operated as described in the '796 patent, using a 60 bar gas source, in order to deliver the insulin particles across the skin and into the Franz cell receiver medium.

In experimental groups receiving an occlusive dressing, the dressing was placed on the skin covering the Franz cell, over the site of particle administration, and left in-place for the duration of the study.

After delivery, samples were withdrawn from the Franz cell and assayed for insulin content using HPLC. Specific study conditions, payload amounts and particle size information are reported with each individual study.

In a first study, the occlusion effect on insulin flux was assessed. Insulin payloads were 3 mg, the particle size fraction used in all groups was 38-53 μm, and the occlusive dressing was prepared from 3-5 mm strips of Parafilm®. The cumulative amounts of delivered insulin (flux) for each experimental group over a 24 hour period are reported in Table 1 below.

TABLE 1

| | Insulin Dose | Ave. Delivery Amount |
|---|---|---|
| Formulation/Treatment | Delivered | (μg/24 hours) |
| Neat Insulin | 3 mg | 116 ± (NA) |
| Neat Insulin/Parafilm | 3 mg | 257 ± 51 |
| Occlusion | | |

As can be seen, the occlusive dressing had a significant enhancing effect on insulin flux.

In a second study, the occlusion effect of different dressing compositions on insulin flux was assessed. Insulin payloads were 3 mg, the particle size fraction used in all groups was 38-53 μm, and the occlusive dressings were prepared from 3-5 mm strips of either Parafilm® or a film of polyisobutlylene (PIB) that was allowed to dry for a 72 hour period. The cumulative amounts of delivered insulin (flux) for each experimental group over a 24 hour period are reported in Table 2 below.

TABLE 2

| | Occlusion | Ave. Delivery Amount |
|---|---|---|
| Formulation | Treatment | (μg/24 hours) |
| Neat Insulin (3 mg) | None | 21 ± 20 |
| Neat Insulin (3 mg) | Parafilm | 184 ± 60 |
| Neat Insulin (3 mg) | PIB | 291 ± 185 |

As can be seen, each occlusive dressing increased insulin flux about 10 times, and the PIB occlusive dressing performed substantially the same as the Parafilm dressing.

In a third study, the occlusion effect of different dressing compositions on insulin flux was again assessed. Insulin payloads were 3 mg, the particle size fraction used in all groups was 38-53 μm, and the occlusive dressings were prepared from 3-5 mm strips of (a) Parafilm®, (b) a film of polyisobutlylene (PIB) that was allowed to dry for a 72 hour period, or (c) a film of polyisobutlylene (PIB) containing 1 mg of neat Porcine insulin that was allowed to dry for a 72 hour period. The cumulative amounts of delivered insulin (flux) for each experimental group over a 24 hour period are reported in Table 3 below.

TABLE 3

| | | Ave. Delivery | |
|---|---|---|---|
| | Occlusion | Amount | |
| Formulation | Treatment | (μg/24 hours) | Range of Flux |
| Neat Insulin (3 mg) | None | 20 ± 7 | 15-25 |
| Neat Insulin (3 mg) | Parafilm | 200 ± 89 | 101-273 |
| Neat Insulin (3 mg) | PIB | 230 ± 126 | 102-352 |
| Neat Insulin (3 mg) | PLB + 1 mg | 612 ± 345 | 243-926 |
| | Insulin | | |

As can be seen, the Parafilm and PIB occlusive dressings significantly improved insulin flux, and the addition of insulin to the occlusive dressing also significantly improved flux relative both to non-occluded and plain occlusion treatment groups.

In a fourth study, the effect of combining occlusive dressings with a modified insulin formulation was assessed. Insulin payloads were 3 mg, the particle size fraction used in all groups was 38-53 μm, and the occlusive dressings were prepared from 3-5 mm strips of a film of polyisobutlylene (PIB) that was allowed to dry for a 72 hour period. In some groups, 30% polyethylene glycol (PEG) was added to the insulin formulation prior to particle formation, while in others, 50% trehalose was added to the insulin formulation prior to particle formation. The cumulative amounts of delivered insulin (flux) for each experimental group over a 24 hour period are reported in Table 4 below.

TABLE 4

| | | Ave. Delivery | |
|---|---|---|---|
| | Occlusion | Amount | |
| Formulation | Treatment | (μg/24 hours) | Range of Flux |
| Neat Insulin (3 mg) | None | 11 ± 1 | 11-12 |
| Neat Insulin (3 mg) | PIB | 250 ± 96 | 156-348 |
| Insulin (3 mg) and | None | 14 ± 13 | 5-29 |
| 30% PEG | | | |
| Insulin (3 mg) and | PIB | 470 ± 164 | 290-610 |
| 30% PEG | | | |
| Insulin (3 mg) and | None | 27 ± 17 | 7-39 |
| 50% trehalose | | | |
| Insulin (3 mg) and | PIB | 257 ± 21 | 233-267 |
| 50% trehalose | | | |

As can be seen, the 30% PEG formulation combined with the PIB occlusive dressing displayed a significantly higher flux than the occlusion-only and PEG-only treatment conditions. Addition of the 50% trehalose provided no benefit over the neat insulin formulations in both occluded and non-occluded treatment conditions.

In a fifth study, the effect of combining occlusive dressings with a modified insulin formulation was assessed over a range of delivery payloads. In addition, to assess whether longer occlusive duration had any beneficial effect, flux was assessed after both 24 and 48 hours of constant occlusion. Insulin payloads were 1, 3, 5 or 7 mg, the particle size fraction used in all groups was 38-53 μm, and the occlusive dressings were prepared from 3-5 mm strips of a film of polyisobutlylene (PIB) that was allowed to dry for a 72 hour period. In all groups, 30% polyethylene glycol (PEG) was added to the insulin formulation prior to particle formation. The cumulative amounts of delivered insulin (flux) for each experimental group over 24 and 48 hour periods are reported in Table 5 below.

TABLE 5

| | | Ave. Delivery | Ave. Delivery |
|---|---|---|---|
| | Occlusion | Amount | Amount |
| Formulation | Treatment | (μg/24 hours) | (μg/48 hours) |
| Insulin (3 mg) and | None | 366 ± 94 | 379 ± 100 |
| 30% PEG | | | |
| Insulin (1 mg) and | PIB | 197 ± 43 | 200 ± 44 |
| 30% PEG | | | |
| Insulin (3 mg) and | PIB | 685 ± 175 | 686 ± 176 |
| 30% PEG | | | |
| Insulin (5 mg) and | PIB | 1006 ± 93 | 1008 ± 94 |
| 30% PEG | | | |
| Insulin (7 mg) and | PIB | 994 ± 72 | 995 ± 72 |
| 50% trehalose | | | |

As can be seen, occlusion with the PIB dressing allowed for almost equivalent delivery amount (flux) from a one-third less dose of insulin. Furthermore, the additional 24 hour occlusion period (48 hour total) provided no benefit for this particular insulin formulation.

### EXAMPLE 2

### Enhanced Flux of Insulin Administered using a Needleless Syringe

In order to assess the ability of an occlusive dressing to enhance the flux of insulin administered via a particle injection device, the following in vivo studies were carried out. A 10% insulin (NaPO₄) composition was obtained from a commercial source and lyophilized, compressed and then milled to produce insulin particles suitable for use in a particle injection device. This particle formation procedure is the same as described above in Example 1. The 38-53 μm sieve fraction was then obtained using a standard mesh screen.

All particle injections were carried out using a PowderJect® model ND1 needleless syringe fitted with a porous silencer (obtained from PowderJect Technologies, Inc., Fremont, Calif.). This device is described in commonly owned U.S. Pat. Nos. 5,630,796 and 6,004,286. Particle payloads (1 mg) were loaded into a seven-piece research drug cassette having 20 μm polycarbonate rupturing membranes. The devices were operated as described in the '796 patent, using a 60 bar gas source, in order to deliver the insulin particles.

Occlusive patches were produced by cutting a Parafilm® disc and holding the same in-place by a Hill-Top chamber. In experimental groups receiving an occlusive dressing, the patch was placed on the skin covering the site of particle administration, and left in-place for the duration of the study.

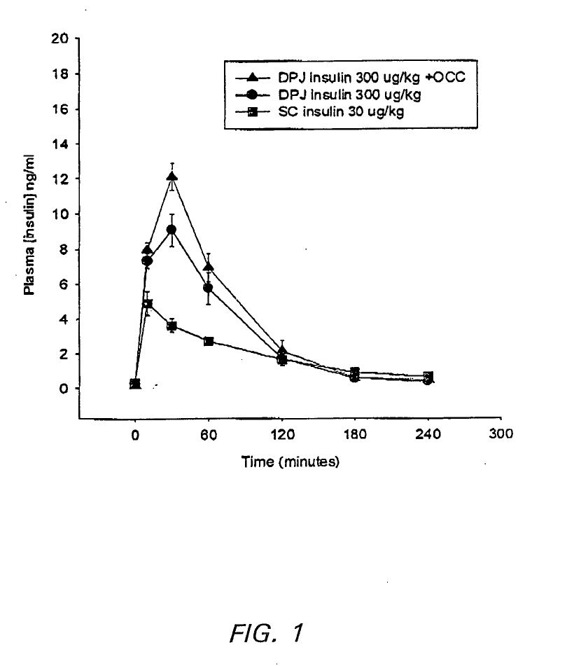

Male Sprague Dawley rats (270-330 g) were anaesthetized and a cannula surgically placed in the right carotid artery. An area (approx 3×3 cm) of the lower right abdomen was clipped and shaved. The particulate insulin dose was administered to the shaved area, and the occlusive patch was immediately placed over the dose site, and left there for the remainder of the study. Arterial blood samples were withdrawn from the carotid artery prior to and at 10, 30, 60, 120, 180, and 240 minutes following the dose. The samples were immediately centrifuged, and the plasma transferred to a new tube and stored at 2-8° C. for no longer than a week prior to analysis. Insulin concentrations were determined from plasma samples by radioimmunoassay according to standard methods. The coefficient of variation for this assay was 12%, 4%, and 8% at 0.3, 4, and 40 ng/ml, respectively. The results of the study are depicted in FIG. 1, wherein “DPJ” indicates particle injection of the particulate insulin composition to the dermis, “OCC” indicates occlusion, and “SC” indicates the subcutaneous (control) administration.

Actual and relative bioavailability (BA) were then calculated according to standard equations using plasma concentration data following subcutaneous (SC) and intravenous (IV) insulin administration, generated in separate studies. The bioavailability values are reported below in Table 6.

TABLE 6

| | Occlusion | | | Actual & |
|---|---|---|---|---|
| Formulation | Treatment | # of Subjects | Mean Dose | (Relative) BA |
| 10% Insulin | None | n = 4 | 287 μg/kg | 11% (16%) |
| (NaPO₄) | | | | |
| 10% Insulin | Parafilm | n = 4 | 298 μg/kg | 14% (20%) |
| (NaPO₄) | | | | |

As can be seen, post-administrative dose-site occlusion promotes systemic uptake of insulin delivered to anaesthetised rats by the PowderJect® model ND1 needleless syringe.

### EXAMPLE 3

### Enhanced Immunogenicity of a Particulate Vaccine Composition Using Occlusion

In order to assess whether the occlusion methods of the present invention improve the immune response to a particulate hepatitis b surface antigen (HbsAg) vaccine composition following epidermal delivery, the following study was carried out

Commercially available Hepatitis B vaccine (adjuvanted with alum) and Hepatitis B surface antigen HbsAg (without alum) were obtained from the Rhein Biotech-Argentine joint venture group. Particulate HbsAg was prepared by formulating the antigen with mannitol/PVP excipients and then freeze-drying to obtain suitable particles. The 38-53 μm sieve fraction was then obtained using a standard mesh screen.

All particle injections were carried out using a PowderJect® model ND 1 needleless syringe fitted with an open-vented spacer (obtained from PowderJect Technologies, Inc., Fremont, Calif.). This device is described in commonly owned U.S. Pat. Nos. 5,630,796 and 6,004,286. Particle payloads (2 mg) were loaded into trilaminate drug cassettes having upper and lower rupturing membranes. The devices were operated as described in the '796 patent, using a 60 bar gas source, in order to deliver the HbsAg particles.

Occlusive patches were produced by cutting 1-1.5 cm diameter Parafilm® discs which were held in-place by athletic tape to avoid previous problems of the patches slipping from position during the course of the study. In experimental groups receiving an occlusive dressing, the patch was placed on the skin covering the site of particle administration after each delivery and left in-place overnight (or until the patches were displaced due to normal animal behavior).

White pigs of mixed sex (approximately 20 lb) were assembled into experimental groups of 5 animals per group. The study was initiated by vaccinating all animals on week 0 using a single intramuscular (IM) injection of one human dose of Hepatitis B vaccine with the alum adjuvant. IM injections were applied to the outside of the upper thigh muscle. The pigs were then boosted on weeks 4 and 8 with the powdered vaccine composition (100 μg of HbsAg at each boost) using the PowderJect needleless syringe as described above. In the group receiving the occlusion treatment, the Parafilm® disks were put in place after each particle administration and left overnight. Control animals were vaccinated with the single IM injection, but occlusion was not used.

Blood samples were collected on week 10, and antibody response to HbsAg was measured using AUSAB assay kit (Abbott Laboratories, Abbott Park, Ill.).

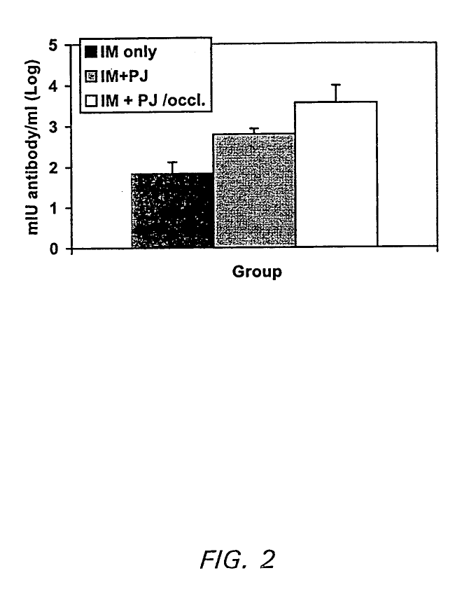

The results of the study are depicted in FIG. 2. As can be seen, the control group (pigs immunized with a single IM injection) had a low level of antibody response on week 10. In the second experimental group (pigs immunized with a single IM priming followed by two boosts with the powdered vaccine composition) showed a 9-fold increase in antibody titer relative to the control group. However, a 52-fold increase was seen in the second experimental group (pigs immunized with a single IM priming followed by two boosts with the powdered vaccine composition and enhanced with the occlusive dressings) relative to the control group. ed animals in antibody titer was observed when occlusion was used. Thus, the animals that received the occlusive treatment showed a 6 fold-higher antibody titer relative to those receiving the powdered boosts only. It is concluded that occlusion can improve the immune response to powdered vaccine composition delivered using a particle injection method.

### EXAMPLE 4

### Clinical Evaluation of Post-Administration Patch Occlusion

In order to evaluate the bioavailability of a peptide drug (calcitonin) administered via particle injection, with and without post administration patch occlusion (relative to subcutaneous injection), the following clinical study was carried out.

For subcutaneous injections, Miacalcic® calcitonin product was obtained from a commercial source and contained approximately 25 μg (100 IU) of salmon calcitonin (sCT) per ml. For particle injections, the calcitonin composition was formed from sCT at 8% w/w, PVP excipient at 2% w/w, in mannitol. The calcitonin composition was then lyophilized, compressed and then milled to produce particles suitable for use in a particle injection device. This particle formation procedure is the same as described above in Example 1. The 38-53 μm sieve fraction was obtained using a standard mesh screen.

All particle injections were carried out using a PowderJect® model ND1 needleless syringe fitted with a porous silencer (obtained from PowderJect Technologies, Inc., Fremont, Calif.). This device is described in commonly owned U.S. Pat. Nos. 5,630,796 and 6,004,286. Particle payloads were loaded into a trilaminate drug cassette having upper and lower rupturing membranes. The devices were operated as described in the '796 patent, using a 60 bar gas source, in order to deliver the sCT particles.

Occlusive patches were produced by cutting Parafilm® discs which were held in-place by an adhesive dressing. In experimental groups receiving an occlusive dressing, the patch was placed on the skin covering the site of particle administration after sCT delivery and left in-place for the duration of the study (4 hours).

The overall approach involved measuring pharmacokinetic and pharmacodynamic response parameters following the sCT dose in 24 healthy female subjects. The trial was designed as a single center, open, parallel group trial. The test matrix is reported below as Table 7.

TABLE 7

| | Route of | Occlusion | Number of | | | Nominal sCT | |
|---|---|---|---|---|---|---|---|
| Formulation | Administration | Treatment | Administrations | Payload | | dose | |
| Miacalcic | subcutaneous | None | 1 | 0.5 | ml | 25 | μg |
| | | | | | | (100 | IU) |
| 8% sCT | particle | None | 3 | 1.25 | mg | 300 | μg |
| | injection | | | | | (1200 | IU) |
| 8% sCT | particle | Parafilm | 3 | 1.25 | mg | 300 | μg |
| | injection | | | | | (1200 | IU) |

n = 8 for each group.

Blood samples were collected via an indwelling catheter prior to and at 2, 5, 10, 20, 30, 45, 60, 90, 120, 180 and 240 minutes following administration of the calcitonin dose. sCT plasma concentrations were determined by ELISA (PPD Development, VA). Plasma ionised calcium (Ca²⁺) concentrations were determined at Covance Laboratories (UK). sCT was detected in the plasma following dose in all subjects. Peak plasma levels were achieved at 5 minutes following particle injection and at 20 minutes following the SC delivery. Assuming nominal doses, mean bioavailability (BA) relative to the SC injection for the particle delivery plus occlusion was 8.5%, while particle delivery without occlusion was 5.1%. Although no significant difference in the area under the curve (AUC) was observed between the three groups (P>0.05, one-way ANOVA), the power of the analysis was low (<0.8). All treatments produced a transient, hypocalcaemic response. There was no significant difference in the mean area above the blood Ca²⁺ concentration when plotted against the time profile between the three treatment groups. Occlusion following the particle delivery of sCT did not affect Cₘₐₓ or Tₘₐₓ, but did appear to affect elimination rate in the 30-240 minute sample time-point range. Since it is extremely unlikely that sCT plasma clearance would be affected by application of the occlusive dressing, the reduction in apparent rate may therefore be attributed to a prolongation of sCT uptake (without affecting absorption rate) into the bloodstream from the site of administration when the occlusive patch was employed.

It was concluded that systemic delivery of sCT was achieved by the particle delivery methods of the present invention, and that post administrative dose site occlusion increased relative bioavailability from 5.1 to 8.5%, most probably by increasing the duration, but not the rate, of sCT systemic uptake.

Accordingly, novel methods for administering a therapeutic agent are disclosed. Although preferred embodiments of the subject invention have been described in some detail, it is understood that obvious variations can be made without departing from the spirit and the scope of the invention as defined by the appended claims.

1.-40. (canceled)

41. A method for administering a therapeutic agent to a predetermined area of skin or mucosa of a vertebrate subject, said method comprising:
(a) accelerating particles into, across or both into and across the area of skin or mucosa, wherein the particles are accelerated toward the skin or mucosa using a needleless syringe device; and
(b) topically positioning a first occlusive dressing over the area of skin or mucosa, wherein the particles comprise the therapeutic agent.

42. The method of claim 41, wherein the particles comprise a placebo.

43. The method of claim 41, wherein step (b) comprises topically positioning the first occlusive dressing over the area of skin or mucosa.

44. The method of claim 41, wherein the particles comprise an antigen.

45. The method of claim 41, wherein the particles comprise an adjuvant.

46. The method of claim 44, wherein the method further comprises a pretreatment step to administer an adjuvant to the area of skin or mucosa before step (a).

47. The method of claim 46, wherein the pretreatment step comprises topically positioning a second occlusive dressing containing an adjuvant over the area of skin or mucosa.

48. The method of claim 44, wherein step (b) comprises topically positioning the first occlusive dressing over the area of skin or mucosa, and further wherein the first occlusive dressing contains an adjuvant.

49. The method of claim 41, wherein the particles comprise a permeation enhancing agent.

50. The method of claim 41, wherein the particles are accelerated toward the skin or mucosal tissue at a velocity of about 200 to 3,000 m/sec.

51. The method of claim 41, wherein the particles have a diameter predominantly in the range of about 0.1 to 250 μm.

52. The method of claim 41, wherein the particles comprise a biologically active protein, a peptide, an oligosaccharide, a polysaccharide or a vaccine composition.

53. The method of claim 41, wherein step (a) provides for rapid delivery onset from the first transdermal delivery device.

54. The method of claim 43, wherein the particles and the first occlusive dressing comprise the same therapeutic agent.

55. The method of claim 42, wherein the placebo comprises particles selected from the group consisting of a metal particle and a metal particle coated with a permeation enhancing agent.

56. The method of claim 55, wherein the particles comprise a permeation enhancing agent.

57. The method of claim 41, further comprising before step (a), topically positioning over the area of skin or mucosa a second occlusive dressing.

58. The method of claim 57, wherein the second occlusive dressing contains a permeation enhancing agent.
